(19) United States
(12) Patent Application Publication
(10) Pub. No.: US 2011/0239538 A1
(43) Pub. Date: Oct. 6, 2011

(54) Environmentally friendly simplified humidifier without power supply

(76) Inventor: Tsang-Hung Hsu, Chia Li (TW)

(21) Appl. No.: 12/798,368

(22) Filed: Apr. 5, 2010

(51) Int. Cl. A01G 9/02; A61L 9/04

(52) U.S. Cl. 47/66.6; 239/44

(57) ABSTRACT

A simplified humidifier includes a holding device having a trough for storing water therein, and a water-absorbable, telescopically sinuously extendible vaporizing element submerged in the trough of the holding device for absorbing water from the trough and spreading the moisture through the vaporizing element to be evaporated into the surrounding for increasing the humidity nearby.

### BACKGROUND OF THE INVENTION

U.S. Pat. No. 7,547,004 also invented by the present inventor disclosed a portable humidifier having an ultrasonic vibrator mounted is a bottle operatively vibrating for ejecting water mists as absorbed through the vibrator for humidifying the environment nearby.

However, such a prior art has the drawbacks as follows:
1. The ultrasonic vibrator must be driven by electricity to consume electric energy.
2. The ultrasonic vibrator and the related elements in construction of the portable humidifier may increase the production cost of the humidifier.
3. The control device and the sensor of the humidifier may increase the operation and maintenance complexity or problems of the humidifier.

The present inventor has found the drawbacks of the prior art and invented the present humidifier without power supply.

### SUMMARY OF THE INVENTION

The object of the present invention is to provide a simplified humidifier including a holding device having a trough for storing water therein, and a water-absorbable, telescopically sinuously extendible vaporizing element submerged in the trough of the holding device for absorbing water from the trough and spreading the moisture through the vaporizing element to be evaporated into the surrounding for increasing the humidity nearby.

Another object of the present invention is to provide a humidifier further including at least a seed planter or planter embedded or kept in the trough of the holding device for providing a true or natural plant as grown from the planter for freshing the interior air and enhancing the decorative effect of the humidifier.

### BRIEF DESCRIPTION OF THE DRAWINGS

FIG. 1 is an illustration showing a humidifier in accordance with the present invention.

FIG. 2 is a longitudinal sectional drawing of the present invention.

FIG. 3 is a sectional drawing as viewed from Line 3-3 of FIG. 2.

FIG. 4 shows a flattened vaporing element when unfolded in accordance with the present invention.

FIG. 5 shows a vaporizing element as folded from FIG. 4.

FIG. 6 is a perspective view of the vaporizing element when sinuously extended in accordance with the present invention.

FIG. 7 is a sectional drawing showing another preferred embodiment of the present invention.

### DETAILED DESCRIPTION

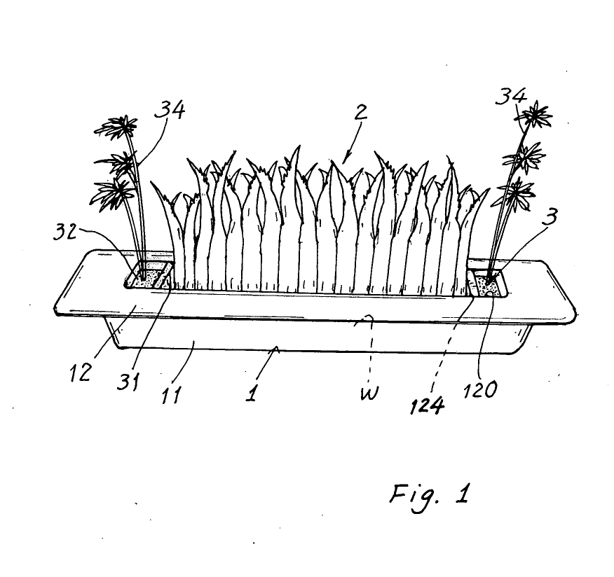

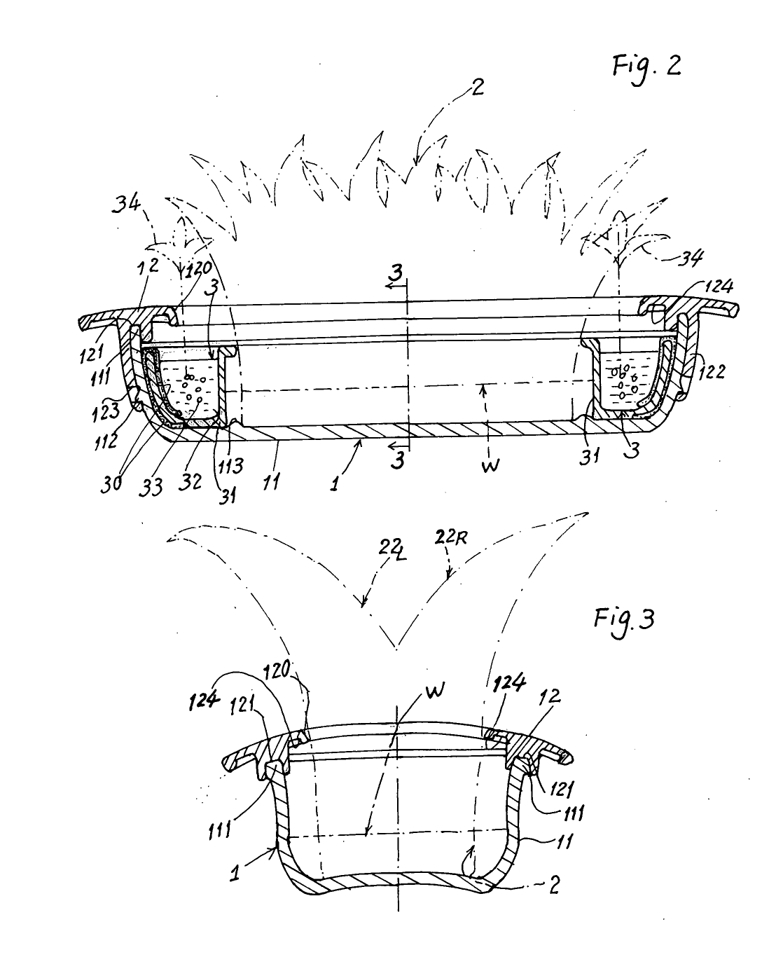

As shown in FIGS. 1˜6, the present invention comprises: a holding device 1, a vaporizing element 2 held and immersed in the holding device 1 for absorbing water or moisture from the holding device 1 and evaporating water vapor to the surrounding for increasing the humidity nearby; and two planters or seed planters 3 held in opposite end portions of the holding device 1.

The planters 3 may be simplified to be a single planter 3 held in the holding device 1.

The holding device 1 may be formed as an elongated shape, a wavy or curved shape, but not limited in the present invention.

The holding device 1 includes: a trough 11 for storing water W in the trough 11, and a cover 12 embedded or covered on an upper portion of the trough 11. The cover 12 includes an opening 120 for protruding the vaporizing element 2 outwardly or upwardly through the opening 120.

The trough 11 includes a top edge portion 111 detachably engaged with a peripheral groove 121 circumferentially formed in a bottom portion of the cover 12 for embedding the cover 12 on the trough 11.

A downwardly-protruded extension 124 is circumferentially formed on the cover 12 along the opening 120 to prevent from outward spillage of water W as stored in the trough 11 when accidentally tilted, thereby preventing from wetting of the surroundings.

The cover 12 further includes two lugs 122 protruding downwardly from opposite ends of the cover 12, end lug 122 formed with a hole 123 therein for engaging a projection 112 sidewardly protruded from the trough 11 to stably engage the cover 12 with the trough 11.

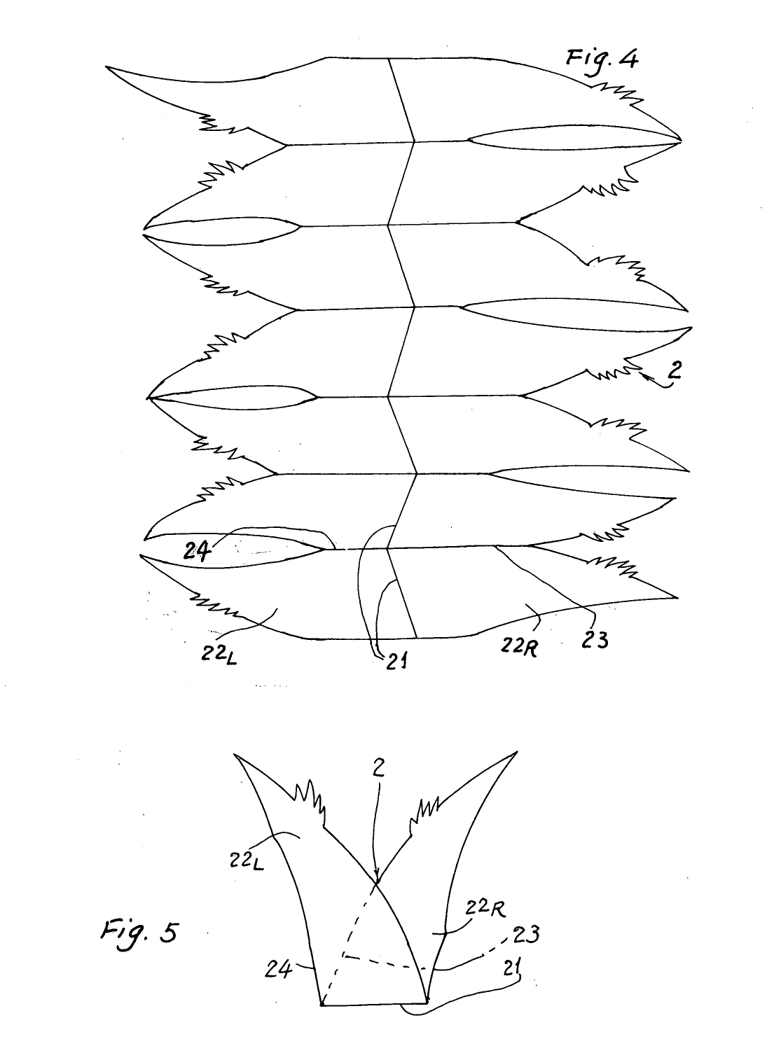

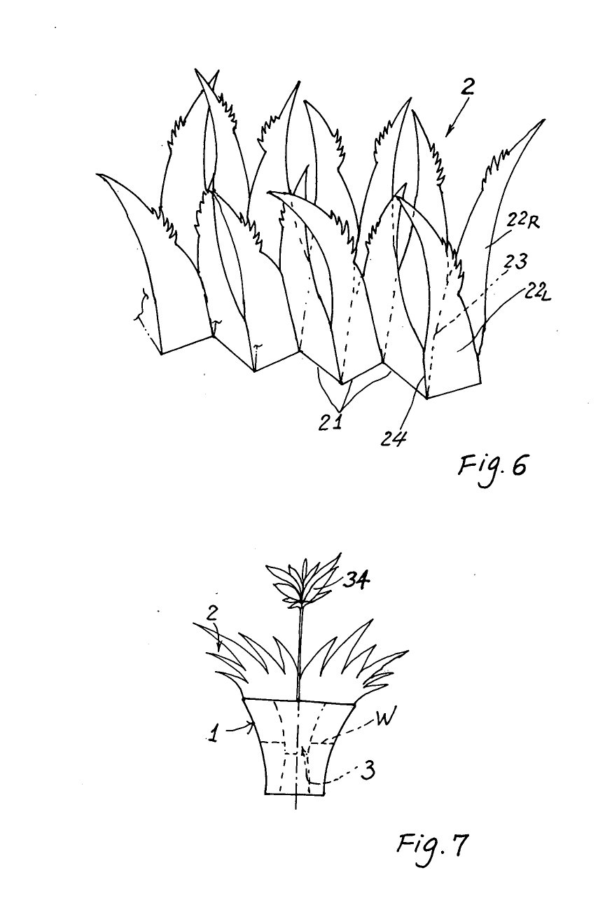

The vaporizing element 2 is made of water absorbable materials including paper and fibrous sheet for capillarily spreading water or moisture through the vaporizing element 2 when immersed in the trough 11 of the holding device 1. The vaporizing element 2 is unfolded and flattened as a sheet or board as shown in FIG. 4. Upon folding or pleating of the vaporizing element 2 to be a three-dimensional shape or form as shown in FIGS. 6 and 1, the vaporizing element 2 will then serve as an evaporator for vaporizing the water to be vapor into the surrounding for increasing the humidity nearby.

The vaporizing element 2 is preferably sinuously or corrugatedly extendible or telescopically retractable along the trough 11 of the holding device 1. Upon extending of the vaporizing element 2 lengthwise in the trough 11, an air-contacting area or diffusing area as effected by the vaporizing element 2 will be increased to increase the total evaporating area of the element 2, thereby increasing the humidity near the vaporizing element 2 as extended.

The longer the vaporizing element 2 is extended, the larger air-contacting area or diffusing area will be, thereby resulting in an increased humidity.

As shown in FIGS. 4˜6, the vaporizing element 4 includes a plurality of right vaporizing leaves 22R and left vaporizing leaves 22L respectively protruding and diverging rightwardly or leftwardly from a bottom holding line 21; with every two neighboring right vaporizing leaves 22R operatively folded along a right folding line 23; and every two neighboring left vaporizing leaves 22L operatively folded along a left folding line 24.

The bottom folding line 21 is formed as a zigzag or corrugated shape and comprised of a plurality of V-shaped line sections 211, each V-shaped line section 211 having an apex 212 formed at a center thereof; each right folding line 23 and each left folding line 24 linearly aligned for intersecting each apex 212 of the V-shaped line section 211 of the bottom folding line 21; each right folding line 23 and each left folding line 24 respectively inclined or flared rightwardly and leftwardly from the bottom folding line 21, thereby diverging the right vaporizing leaves 22R and left vaporizing leaves 22L rightwardly and leftwardly to mimic true plant leaves, bracken, grass or flowers, thereby increasing the diffusing or vaporizing area of the vaporizing element 2, besides the decorative effect thereof.

The vaporizing element 2 may be extendibly folded or pleated along the bottom folding line 21, the right folding lines 23 and the left folding lines 24 to be sinuously extended in the trough 11 of the holding device 1 to form an imitated tree or plant as shown in FIG. 1 for interior decoration; with the extending of the vaporizing leaves 22R, 22L, the air-contacting area or diffusing area of the vaporizing element 2 will be increased to enhance the evaporation of the water or moist as absorbed and spread through the vaporizing element 2 into the surrounding to thereby increase the humidity nearby.

No power (electricity) consumption, low cost of vaporizing element 2 such as made of paper, and simple construction of the humidifier, an environmentally friendly humidifier may be made in accordance with the present invention, which is simpler, cheaper and more environmentally friendly than the prior art.

Before being used, the vaporizing element 2 is folded or retracted as shown in FIG. 5 to minimize its volume for convenient storage and handling.

Although the vaporizing element 2 as illustrated is formed to mimic plant leaves, bracken, grass or flowers, other shapes or designs may however be modified, not limited in the present invention.

For instance, the vaporizing element 2 may be formed as a mountain or architecture model, human or animal gathering, doll assembly, decorative or advertising feature, regularly or irregularly-shaped design, not limited in the present invention. The holding device 1 may also be modified to be other art or decorative designs, other than the elongated trough as shown in FIGS. 1˜3.

The present invention further comprises a planter 3 fixed, embedded or placed in the trough 11 of the holding device 1.

As shown in FIG. 1, there are two planters 3 respectively disposed in opposite end portions of the holding device 1.

Each planter 3 includes: a pot 31 placed in the trough 11 of the holding device 1; a wick element 30 having its first end protruding into a bottom portion of the trough 11 to absorb water W as stored in the trough 11, and having a second end of the wick element 30 inserted into the bottom portion of the pot 31 to be communicated with a soil, planting medium or nutrient 32 stored in the pot 31 for capillarily feeding water into the pot 31, and a plant 34 such as flower, bracken, tree or any home plant planted in the pot 31 to absorb water and nutrient from the pot 31 for its growth.

The planter 3 may also include: a plurality of seeds 33 buried in the soil 32 in the pot 31, whereby upon absorbing water and nutrient from the pot 31, the seeds 33 may be grown up to be plants 34 as illustrated in FIG. 2. The planter 3 may be embedded and limited by a bottom protrusion 113 formed in the trough 11.

The planter 3, as pre-buried with seeds 33 therein, should be sealed with thin film for convenient storage, shipping and handling thereof.

As shown in FIG. 7, the holding device 1 is modified to be a cylindrical pot having the planter 3 placed in a central portion of the holding device 1, whereby the plants or flowers 34 will be surrounded by vaporizing element 2 which may be formed as plant leaves, to form a home planter for enhancing decorative effect and increasing humidity in the home interior.

The present invention may be provided in a bed room; an office, a living room, and other interiors for both interior decoration and humidification for enhancing the living environment to be more artistic and healthy.

In a practical experiment, a trough 11 of the holding device 1 of the present invention with a size of 30 cm length×10 cm width×8 cm depth having water stored in the trough is kept in a small room having a dimension of 4 m length×4 m width×2 m height for testing. The vaporizing element 2 for test is made of paper, having a 2 D dimension of about 30 cm×20 cm as flattened before being folded.

The room temperature is kept at 20° C., and the relative humidity is measured to be 15% just at the beginning when the humidifier of the present invention is placed in the room.

Then, the humidifier is placed in the room for humidification overnight as natural evaporation by the paper-made vaporizing element 2 of the present invention. After overnight, it is measured to obtain a relative humidity of 45%, indicating an increase of 30% humidity as effected by the present invention.

According to the present invention, the humidity may be increased up to about 20˜35% in a room provided with the humidifier of the present invention.

Even the vaporing element 2 is formed as an artificial plant. The true or natural plants 34 of the planters 3 as juxtapositioned to the artificial plant of the vaporizing element 2 may activate the artificial plant of the vaporizing element 2 to provide a small interior ecosystem or a “miniature garden” just in the office or in a room, as humidified and decorated synergetically.

Since the present invention does not consume electricity and the elements in construction of the humidifier are simple with lower cost, it is really environmentally friendly or protective. The water vapor is naturally evaporated, without causing excess moisture content in the air to therefore be superior to a conventional electrically operated humidifier.

If needed, a perfume or air refreshing agent may be further added into the trough of the present invention for rendering perfuming or refreshing environment.

For commercial purpose, the planter 3 may also be pre-buried with seeds 33 in the pot 31 which is then sealed with thin film for convenient or hygienic shipping, storage and handling. When used, the planter 3 (by removing the film) is then placed into the trough 11 for absorbing water from the trough for watering and growing the seeds to become plants 34.

The present invention may be further modified without departing from the spirit and scope 11 the present invention.

I claim:

1. A simplified humidifier comprising:
a holding device having a trough stored with water therein; and
a vaporing element made of water absorbable materials held in said trough and submerged in the water for absorbing the water and capillarily spreading the water through said vaporizing element to be naturally evaporated into the surrounding air to increase the humidity nearby.

2. A humidifier according to claim 1, wherein said holding device includes a cover for covering said trough, having an opening formed in said cover for protruding said vaporizing element outwardly through said opening in said cover.

3. A humidifier according to claim 2, wherein said trough includes a top edge portion detachably engaged with a groove circumferentially formed in a bottom portion of said cover for engaging said cover on said trough.

4. A humidifier according to claim 2, wherein said cover further includes an extension circumferentially formed on said cover along said opening of said cover to prevent from outward spillage of water as stored in said trough.

5. A humidifier according to claim 2, wherein said cover includes at least a lug having a hole formed in said lug to be engaged with a projection formed on said trough for stably engaging said cover on said trough.

6. A humidifier according to claim 1, wherein said vaporizing element is selected from paper and fibrous materials for capillarily absorbing water from said trough, and spreading water through said vaporizing element for water evaporation from said vaporizing element.

7. A humidifier according to claim 1, wherein said vaporizing element includes a plurality of right vaporizing leaves and left vaporizing leaves respectively protruding and diverging rightwardly or leftwardly from a bottom holding line; with every two neighboring right vaporizing leaves operatively folded along a right folding line; and every two neighboring left vaporizing leaves operatively folded along a left folding line.

8. A humidifier according to claim 7, wherein said bottom folding line is formed as a zigzag or corrugated shape and comprised of a plurality of V-shaped line sections, each V-shaped line section having an apex formed at a center thereof; each said right folding line and each said left folding line linearly aligned for intersecting each said apex of the V-shaped line section of the bottom folding line; each said right folding line and each said left folding line respectively inclined or flared rightwardly and leftwardly from the bottom folding line, thereby diverging the right vaporizing leaves and left vaporizing leaves rightwardly and leftwardly to mimic true plant leaves.

9. A humidifier according to claim 1, wherein said holding device further includes at least a planter fixed or embedded in the trough of the holding device.

10. A humidifier according to claim 9, wherein said planter includes:
a pot placed in the trough of the holding device; a wick element having a first end of said wick element protruding into a bottom portion of the trough to absorb water as stored in the trough, and having a second end of the wick element inserted into a bottom portion of the pot to be communicated with a soil or nutrient stored in the pot for capillarily feeding water into the pot, and a plant planted in the pot to absorb water and nutrient from the pot for growing the plant.

11. A humidifier according to claim 9, wherein said planter includes:
a plurality of seeds buried in soil and nutrient stored in the pot, whereby upon absorbing water and nutrient from the pot, the seeds will be grown up to be plants.

12. A humidifier according to claim 11, wherein said planter, as pre-buried with seeds therein, is sealed with a thin film for shipping and storage.

13. A humidifier according to claim 1, wherein said vaporizing element is formed as a decorative feature inserted in said trough.
